(19) United States
(12) Patent Application Publication
(10) Pub. No.: US 2026/0000519 A1
(43) Pub. Date: Jan. 1, 2026

(54) Atlantoaxial Prosthetic Joint

(71) Applicant: University of Cincinnati, Cincinnati, OH (US)

(72) Inventors: Botao Zhang, Cincinnati, OH (US); Manish Raj Aryal, Cincinnati, OH (US); Sundararaman Anand, West Chester, OH (US); Eric Allen Nauman, Cincinnati, OH (US); Jonathan Forbes, Cincinnati, OH (US); Abhijith Matur, Cincinnati, OH (US); Katie Phillips, Cincinnati, OH (US)

(73) Assignee: University of Cincinnati, Cincinnati, OH (US)

(21) Appl. No.: 18/992,233

(22) PCT Filed: Jul. 10, 2023
(86) PCT No.: PCT/US2023/027299

§ 371 Date: Jan. 8, 2025

Related U.S. Application Data
(60) Provisional application No. 63/359,340, filed on Jul. 8, 2022.

(51) Int. Cl. A61F 2/44; A61F 2/30

(52) U.S. Cl. CPC A61F 2/44; A61F 2002/30677; A61F 2310/00023

(57) ABSTRACT

The present invention involves a prosthetic device for supporting an atlantoaxial joint between a C1 vertebra and a C2 vertebra after an odontoidectomy procedure. The device includes a prosthetic dens replacement which is fixed to the C2 vertebra via at least two screws. It also includes a bracket to replace the anterior arch of the C1 vertebra. The bracket is affixed to lateral masses of the C1 vertebra via at least two screws. In addition, the bracket, when affixed to the C1 vertebra, is engageable with the dens replacement that is affixed to the C2 vertebra.

### CROSS REFERENCE TO RELATED APPLICATIONS

This application claims priority to U.S. Provisional Application Ser. No. 63/359,340, filed Jul. 8, 2022, which application is hereby incorporated by reference in its entirety.

### TECHNICAL FIELD

The present invention relates to a method and device for treating occipitocervical and atlantoaxial arthrodesis. More specifically, this invention relates to an endoscopic endonasal surgical approach for implanting an atlantoaxial prosthetic joint.

### BACKGROUND OF THE INVENTION

Surgical techniques for occipitocervical and atlantoaxial arthrodesis are used to treat instability involving the occipitocervical (O-C1) and atlantoaxial (C1-2) joints following odontoidectomy. However, this often requires a separate incision to be made to perform fusion and fixation via a posterior approach and results in a loss of mobility, especially lateral rotation of the head. Implantation of an atlantoaxial prosthetic device could help to relieve ventral brainstem compression while preserving motion and reducing the time spent under anesthesia. It would be advantageous to implant such a device through the endonasal corridor. To date, however, no validated method for implanting an atlantoaxial prosthetic device in such a manner exists.

### SUMMARY OF THE INVENTION

The present invention is a novel endonasal method for implanting an atlantoaxial prosthetic joint. One embodiment of the present invention is a system that enables a treatment for occipitocervical and atlantoaxial arthrodesis. This new method provides a minimally invasive advance in the ability of surgeons to preserve motion of the atlantoaxial joint after an odontoidectomy. In one embodiment, the present invention involves a prosthetic device for supporting an atlantoaxial joint between a C1 vertebra and a C2 vertebra after an odontoidectomy procedure resulting in a C1 vertebra without an anterior arch and a C2 vertebra without a dens. The device includes a prosthetic dens replacement which is capable of being fixed to the C2 vertebra via at least two screws. It also includes a bracket to replace the anterior arch of the C1 vertebra. The bracket is capable of being affixed to lateral masses of the C1 vertebra via at least two screws. In addition, the bracket, when affixed to the C1 vertebra, is engageable with the dens replacement that is affixed to the C2 vertebra.

In one embodiment, the prosthetic dens replacement is capable of being fixed to the C2 vertebra via at least two cantilevered screws. In another embodiment, the prosthetic dens replacement comprises titanium. In one embodiment, the bracket comprises titanium. In another embodiment, the prosthetic dens replacement and the bracket comprise an antibiotic coating.

In another aspect of the present invention, a method for implanting a prosthetic dens in a C2 vertebra is provided. The method involves performing an endoscopic endonasal odontoidectomy procedure wherein the patient's anterior arch is removed from the patient's C1 vertebra and the patient's dens is removed from the patient's C2 vertebra. Then, a prosthetic dens replacement is placed through a patient's endonasal corridor. The prosthetic dens replacement is positioned and fixed the to the patient's C2 vertebra where the dens was previously. After this, a bracket is placed through a patient's endonasal corridor. The bracket is positioned and fixed to the patient's C1 vertebra where the anterior arch of C1 was previously. Finally, the bracket is engaged with the prosthetic dens replacement.

In one embodiment, the odontoidectomy procedure involves first exposing the patient's O-C1 joint through the endonasal corridor using an inferior U-shaped nasopharyngeal flap. Then, the anterior arch of the patient's C1 vertebra is removed and the dens of the patient's C2 vertebra is removed.

In another embodiment, the dens replacement is fixed to the C2 vertebra via at least two cantilevered screws. In one embodiment, the bracket is fixed to the C1 vertebra via at least two screws. In another embodiment, the bracket is fixed to the C1 vertebra using neuronavigation guidance. In one embodiment, the prosthetic dens replacement comprises titanium. In another embodiment, the bracket comprises titanium. In one embodiment, the prosthetic dens replacement and the bracket comprise an antibiotic coating.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1A is a perspective view of one embodiment of the anterior arch bracket and dens replacement of the present invention.

FIG. 1B is a perspective view of one embodiment of the anterior arch bracket of the present invention.

FIG. 1C is a perspective view of one embodiment of the dens replacement of the present invention.

FIG. 2 is an image showing occipitocervical fixation.

FIG. 3A is an expanded image of C1-C2 before segmentation.

FIG. 3B is an expanded image of C1-C2 after segmentation.

FIG. 4 is an image showing an anterior view of C1.

FIG. 5 is an image showing a top view of C1.

FIG. 6 is a perspective view of C2, showing dimensional lines for the odontoid process.

FIG. 7A is a perspective view of a first embodiment of the anterior arch bracket of the present invention.

FIG. 7B is a perspective view of a second embodiment of the anterior arch bracket of the present invention.

FIG. 8A is a perspective view of a first embodiment of the dens replacement of the present invention.

FIG. 8B is a perspective view of a second embodiment of the dens replacement of the present invention.

FIG. 8C is a perspective view of a third embodiment of the dens replacement of the present invention.

FIG. 8D is a perspective view of a fourth embodiment of the dens replacement of the present invention.

FIG. 9 is an image of a bracket according to the present invention attached to a C1.

FIG. 10 is an image of a dens replacement according to the present invention attached to a C2 after an odontoidectomy.

### DETAILED DESCRIPTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

Certain terminology is used in the following description for convenience only and is not limiting. The words “right”, “left”, “lower” and “upper” designate directions in the drawings to which reference is made. The words “inwardly” or “proximally” and “outwardly” or “distally” refer to directions toward and away from, respectively, the geometric center of the implant and related parts thereof. The words, “anterior”, “posterior”, “superior,” “inferior” and related words and/or phrases designate preferred positions and orientations in the human body to which reference is made and are not meant to be limiting. The terminology includes the above-listed words, derivatives thereof and words of similar import.

Similar reference numerals will be utilized throughout the application to describe similar or the same components of each of the preferred embodiments of the implant described herein and the descriptions will focus on the specific features of the individual embodiments that distinguish the particular embodiment from the others

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, micro discectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, muscle, ligaments, tendons, cartilage and/or bone unless specifically referred to otherwise.

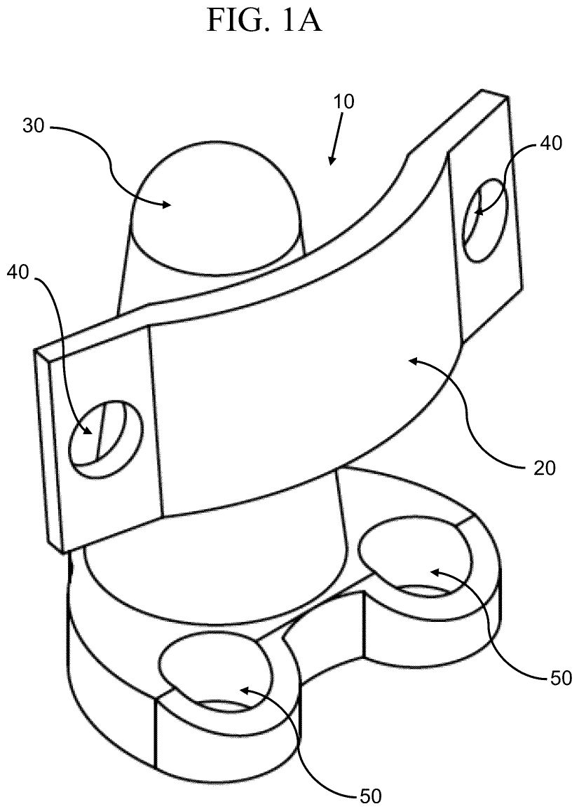

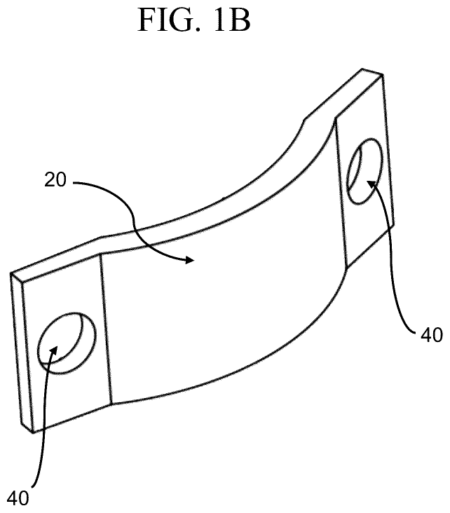

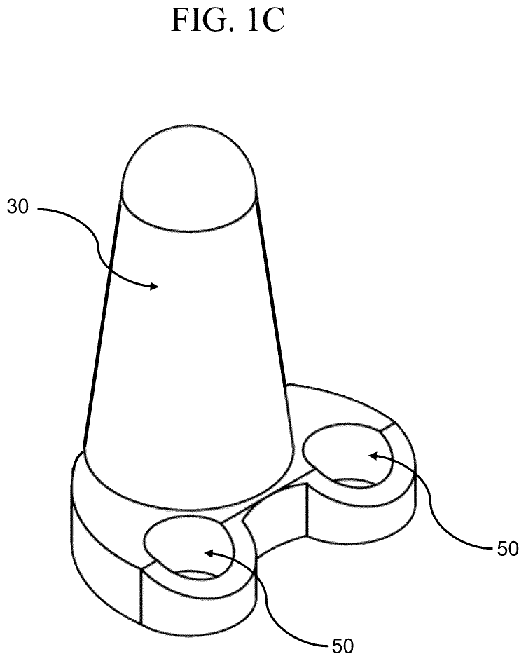

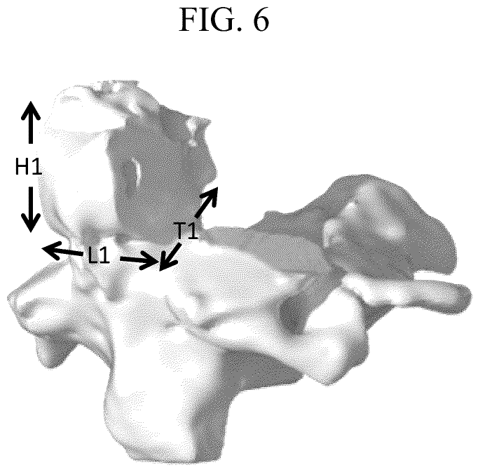

The following discussion includes a description of a prosthetic device and related methods of employing the prosthetic device in accordance with the principles of the present disclosure. Alternate embodiments are also disclosed. Reference is made in detail to the exemplary embodiments of the present disclosure, which are illustrated in the accompanying figures. Turning to FIGS. 1-10, there are illustrated components of a prosthetic device, such as, for example, a system as shown in FIG. 1A.

Surgical techniques for occipitocervical and atlantoaxial arthrodesis are used to treat instability involving the occipitocervical (O-C1) and atlantoaxial (C1-2) joints following odontoidectomy. However, this often requires a separate incision to be made to perform fusion and fixation via a posterior approach and results in a loss of mobility, especially lateral rotation of the head. Implantation of an atlantoaxial prosthetic device could help to relieve ventral brainstem compression while preserving motion and reducing the time spent under anesthesia. Referring to FIGS. 1A-1C, the present invention involves a prosthetic device 10 comprising a prosthetic dens replacement 30 which is fixed to the body of C2 via two cantilevered screws (not shown) inserted through screw holes 50. The prosthetic device 10 also comprises a bracket 20 to replace the anterior arch of C1 affixed to the lateral masses of C1 via two screws (not shown) inserted through bracket screw holes 40. These components can be placed through the endonasal corridor following endoscopic endonasal odontoidectomy. Both components have a small enough profile to pass through the endonasal corridor. Once placed, the device will reconstruct the atlantoaxial joint by replacing the bony elements which were removed during odontoidectomy.

The device of the present invention provides a means to allow for ventral brainstem decompression while retaining mobility at the craniocervical junction in patients needing an odontoidectomy. Placement of this prosthetic through a minimally invasive corridor is expected to help minimize pain, scarring, hospital length of stay, and post-operative disability. The ability to preserve motion of the atlantoaxial joint may also prove beneficial to patients from a quality-of-life perspective (e.g. being able to change lanes safely by looking at blind spots while driving). The present invention can be used in patients in need of odontoidectomy. In this population, the instrumentation makes it possible for the patient to be treated via a single-stage approach instead of requiring a second procedure for posterior arthrodesis. Collectively, these advances will decrease pain, disability, and requisite operative time with expected, attendant decreases in patient morbidity and preserved quality of life.

### Method of Use

In one embodiment, the surgical system of the present invention is implanted as follows. The O-C1 joint is first exposed through the endonasal corridor using the inferior U-shaped nasopharyngeal flap. The anterior arch of C1 and the dens are then removed. Next, the dens replacement is positioned and fixed to the body of C2 with two cantilevered screws. Following this, the bracket is positioned where the anterior arch of C1 was previously and fixed in position with two C1 lateral mass screws placed under neuronavigation guidance.

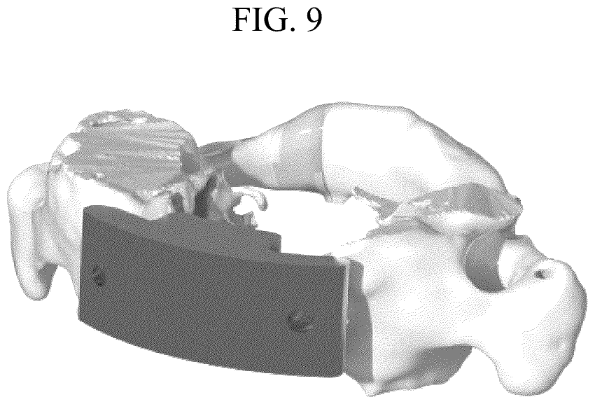

In one embodiment, the dens replacement and C1 bracket are comprised of titanium or similar biocompatible material. Additionally, the dens replacement and C1 bracket may also have an antibiotic coating to reduce the risk of infection resulting from the clean-contaminated corridor. The dens replacement is designed to mimic the shape and proportions of the dens while retaining a small enough footprint to be placed through the endonasal corridor. Static and fatigue loading analyses may be performed to make the bracket and dens design robust and last the lifetime of the patient. The anterior arch bracket is similarly designed to reconstruct the anterior arch of C1. By reconstructing the bony elements that were removed during odontoidectomy, the joint itself will be reconstructed (see FIGS. 1B and 9).

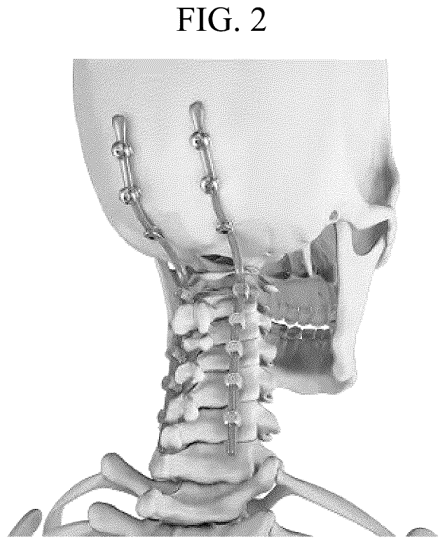

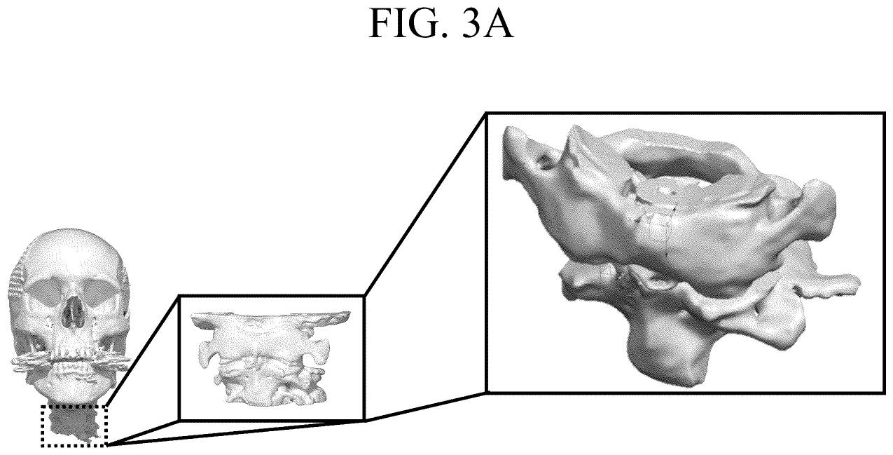

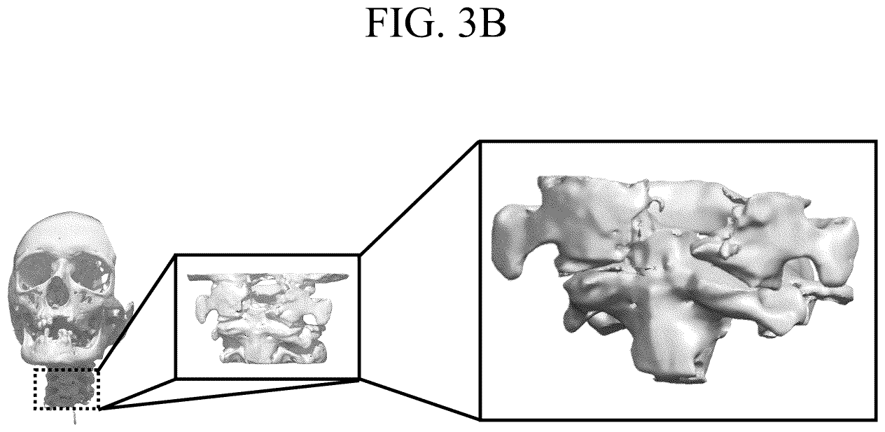

The current standard for treating irreducible brain stem compression is shown in FIG. 2. The figure shows an occipitocervical fixation after an odontoidectomy. Such a procedure results in a 40% loss of flexion-extension and a 60% loss of axial rotation. FIG. 3A shows an expanded image of C1-C2 before segmentation. FIG. 3B shows the C1-C2 after segmentation.

FIG. 1B shows an embodiment of the anterior arch bracket of the present invention. FIG. 1C shows the dens replacement and FIG. 1A shows the system in its configuration after placement. The dens replacement is fixed to the body of C2 followed by the anterior arch bracket, which is attached to the lateral masses of C1 via two lateral mass screws. This configuration allows for flexion, extension, and lateral rotation at the atlantoaxial joint, thereby preserving motion. Rigid fixation of the implants would prevent instability.

The length of the screws used to attach the bracket and the dens replacement are predetermined by individual patient anatomy.

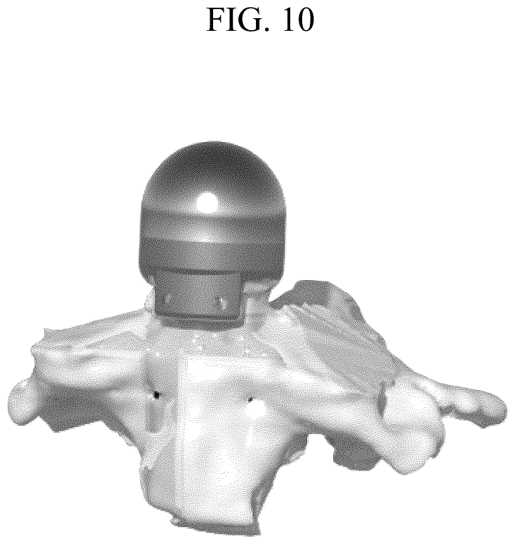

FIG. 9 is an image of a bracket according to the present invention attached to a C1. FIG. 10 is an image of a dens replacement according to the present invention attached to a C2 after an odontoidectomy.

### EXAMPLES

### Example 1

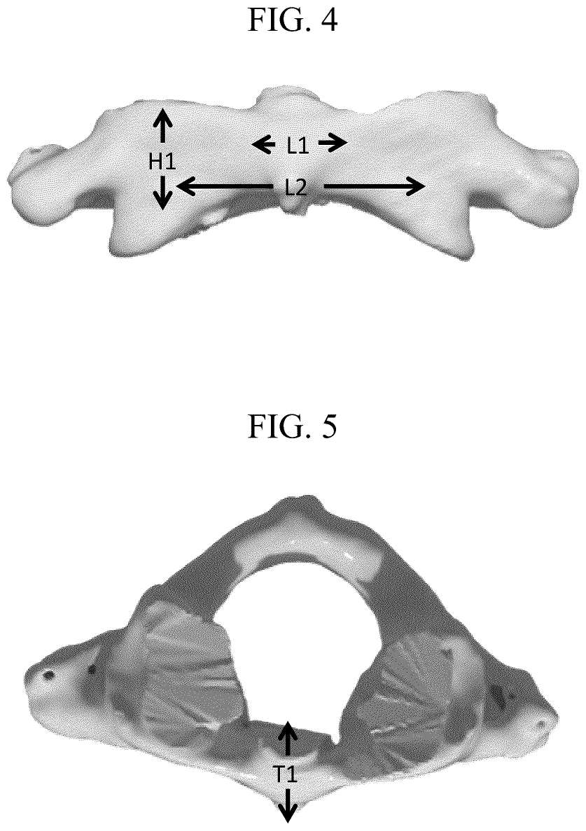

Four cadaveric specimens were examined to determine average dimensions of the anterior arch of C1 and the dens of C2. C1 and C2 were accessed using endonasal inverted U-shaped nasopharyngeal flaps. FIGS. 4 and 5 show the dimensions of C1 used for the analysis. For the C1 measurements, H1 is the height of the C1 anterior arch, T1 is the thickness of the C1 anterior arch, L1 is the average distance across the entire ridge (determined using polygon modeling with Siemens NX software). L2 is the horizontal distance of the C1 anterior presentation. Table 1 shows the measurements taken from the four specimens.

TABLE 1
C1 Measurements

| | H1 | T1 | L1 | L2 |
|---|---|---|---|---|
| Specimen 1 | 14.7 | 8.07 | 19.57 | 26.54 |
| Specimen 2 | 14.68 | 9.96 | 17.95 | 29.17 |
| Specimen 3 | 14.67 | 8.51 | 15.85 | 24.05 |
| Specimen 4 | 13.03 | 8.26 | 17.83 | 29.57 |

For the C2 measurements, H1 is 70% of the height of the odontoid before surgery. The lowest point was determined as widest portion of curvature before LMs. T1 is the thickness of the C2. L1 is the horizontal distance of bony removal of C1 necessary to perform odontoidectomy.

Table 2 shows the measurements taken from the four specimens.

TABLE 2
C2 Measurements

| | H1 | T1 | L1 |
|---|---|---|---|
| Specimen 1 | 12.098 | 17.039 | 16.143 |
| Specimen 2 | 11.345 | 18.405 | 10.321 |
| Specimen 3 | 12.326 | 17.009 | 9.056 |
| Specimen 4 | 13.613 | 15.2 | 11.216 |

### Example 2

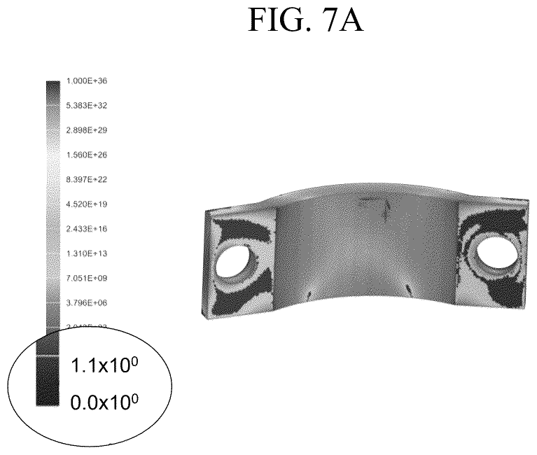

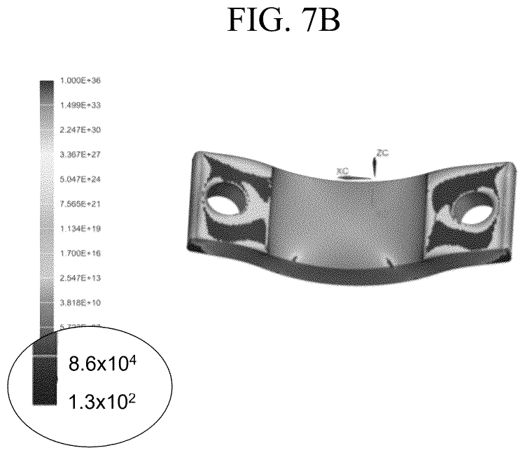

Two embodiments of the bracket of the present invention were formed and tested. Embodiment 1 is shown in FIG. 7A. The figure shows an image of the bracket. The elevation of different portions of the bracket are indicated by shading with the values detailed in the adjacent chart. Embodiment 2 is shown in FIG. 7B, with a similar image of the bracket and adjacent table of values. These brackets were tested for a variety of parameters, including deformation, stress, reaction force, fatigue life, and safety factor. As shown in Table 3, embodiment 2 of the bracket performed better for all parameters.

TABLE 3
C1 Bracket Embodiments

| Parameter | Embodiment 1 | Embodiment 2 |
|---|---|---|
| Deformation | 2 | 1 |
| Stress | 2 | 1 |
| Reaction Force | 2 | 1 |
| Fatigue Life | 2 | 1 |
| Safety Factor | 2 | 1 |

### Example 3

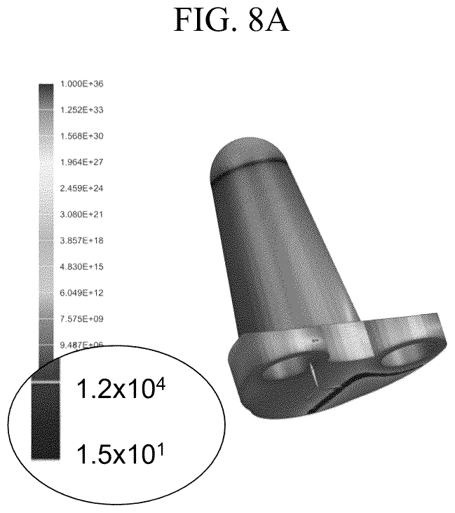

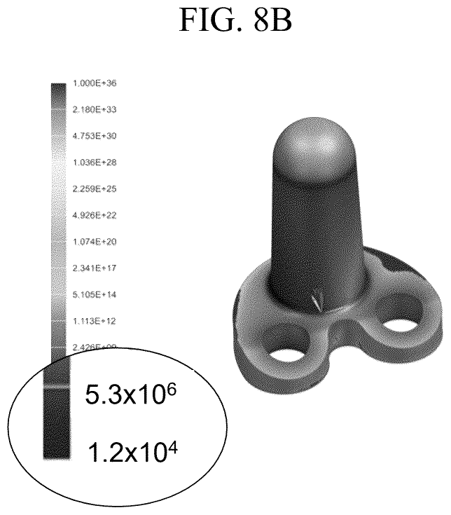

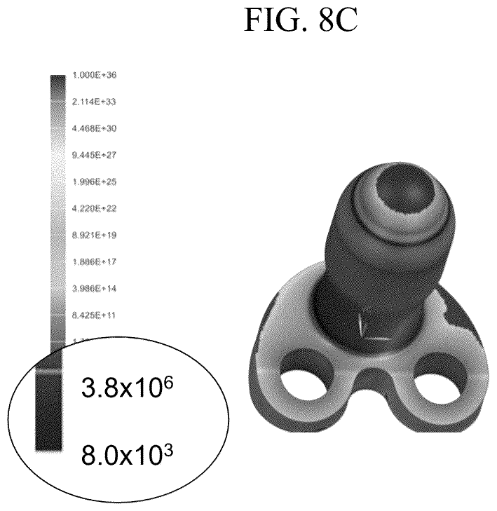

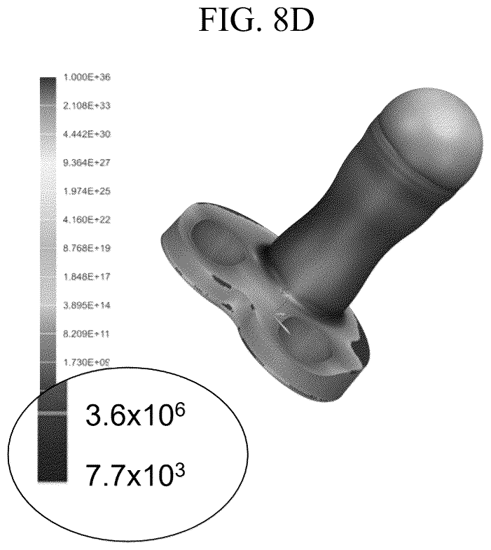

Four embodiments of the prosthetic dens replacement of the present invention were formed and tested. Embodiment 1 is shown in FIG. 8A. The figure shows an image of the dens replacement. The elevation of different portions of the dens replacement are indicated by shading with the values detailed in the adjacent chart. Embodiment 2 is shown in FIG. 8B, with a similar image of the dens replacement and adjacent table of values. Embodiment 3 is shown in FIG. 8C and embodiment 4 is shown in FIG. 8D. These dens replacements were tested for a variety of parameters, including deformation, stress, reaction force, fatigue life, and safety factor. Based on the values shown in Table 4, embodiment 2 was determined to be the dens replacement with the best average performance over all the parameters.

TABLE 4
C2 Dens Replacement Embodiments

| | Embodi- | Embodi- | Embodi- | Embodi- |
|---|---|---|---|---|
| Parameter | ment 1 | ment 2 | ment 3 | ment 4 |
| Deformation | 1 | 3 | 2 | 4 |
| Stress | 4 | 1 | 2 | 3 |
| Reaction Force | 4 | 2 | 3 | 1 |
| Fatigue Life | 4 | 1 | 2 | 3 |
| Safety Factor | 4 | 3 | 1 | 2 |

Having shown and described various embodiments of the present invention, further adaptations of the methods and systems described herein may be accomplished by appropriate modifications by one of ordinary skill in the art without departing from the scope of the present invention. For instance, the examples, embodiments, geometrics, materials, dimensions, and the like discussed above are illustrative and are not required. Accordingly, the scope of the present invention should be considered in terms of the following claims and is understood not to be limited to the details of structure and operation shown and described in the specification and drawings.

What is claimed is:

1. A prosthetic device for supporting an atlantoaxial joint between a C1 vertebra and a C2 vertebra after an odontoidectomy procedure resulting in a C1 vertebra without an anterior arch and a C2 vertebra without a dens, the device comprising:
a. a prosthetic dens replacement which is capable of being fixed to the C2 vertebra via at least two screws; and;
b. a bracket to replace the anterior arch of the C1 vertebra, wherein the bracket is capable of being affixed to lateral masses of the C1 vertebra via at least two screws;
wherein the bracket, when affixed to the C1 vertebra, is engageable with the dens replacement that is affixed to the C2 vertebra.

2. The prosthetic device of claim 1 wherein the prosthetic dens replacement is capable of being fixed to the C2 vertebra via at least two cantilevered screws.

3. The prosthetic device of claim 1 wherein the prosthetic dens replacement comprises titanium.

4. The prosthetic device of claim 1 wherein the bracket comprises titanium.

5. The prosthetic device of claim 1 wherein the prosthetic dens replacement and the bracket comprise an antibiotic coating.

6. A method for implanting a prosthetic dens in a C2 vertebra, comprising:
a. performing an endoscopic endonasal odontoidectomy procedure wherein the patient's anterior arch is removed from the patient's C1 vertebra and the patient's dens is removed from the patient's C2 vertebra;
b. placing the prosthetic dens replacement of claim 1 through a patient's endonasal corridor;
c. positioning and fixing the prosthetic dens replacement to the patient's C2 vertebra where the dens was previously;
d. placing the bracket of claim 1 through a patient's endonasal corridor;
e. positioning and fixing the bracket to the patient's C1 vertebra where the anterior arch of C1 was previously; and
f. engaging the bracket with the prosthetic dens replacement.

7. The method of claim 6 wherein the odontoidectomy procedure comprises:
a. exposing the patient's O-C1 joint through the endonasal corridor using an inferior U-shaped nasopharyngeal flap; and
b. removing the anterior arch of the patient's C1 vertebra and the dens of the patient's C2 vertebra.

8. The method of claim 6 wherein the dens replacement is fixed to the C2 vertebra via at least two cantilevered screws.

9. The method of claim 6 wherein the bracket is fixed to the C1 vertebra via at least two screws.

10. The method of claim 9 wherein the bracket is fixed to the C1 vertebra using neuronavigation guidance.

11. The method of claim 6 wherein the prosthetic dens replacement comprises titanium.

12. The method of claim 6 wherein the bracket comprises titanium.

13. The method of claim 6 wherein the prosthetic dens replacement and the bracket comprise an antibiotic coating.
